(19) United States
(12) Patent Application Publication
(10) Pub. No.: US 2004/0253216 A1
(43) Pub. Date: Dec. 16, 2004

(54) Activated checkpoint therapy and methods of use thereof

(76) Inventor: Chiang J. Li, West Roxbury, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/887,009

(22) Filed: Jul. 8, 2004

Related U.S. Application Data
(63) Continuation of application No. 10/622,854, filed on Jul. 17, 2003.
(60) Provisional application No. 60/396,360, filed on Jul. 17, 2002.

(51) Int. Cl. A61K 48/00; A61K 39/395

(52) U.S. Cl. 424/93.21; 424/155.1

(57) ABSTRACT

Disclosed herein are novel methods and compositions for Activated Checkpoint Therapy™. Also disclosed are methods of treating cancer and apoptosis-associated disorders using cell cycle checkpoint activation modulators. The invention further discloses methods for screening for cell cycle checkpoint activation modulators and the cell cycle checkpoint activation modulators identified by those screening methods.

### RELATED APPLICATIONS

The present patent application is a continuation of U.S. Ser. No. 10/622,854, filed Jul. 17, 2003, which claims the benefit of U.S. Ser. No. 60/396,360, filed Jul. 17, 2002. The contents of these applications are incorporated herein by reference in their entireties.

### FIELD OF THE INVENTION

This invention relates generally to methods and compositions for the regulation of the cell cycle and apoptosis. More particularly, the invention relates to the transient activation of one or more cell cycle checkpoints, activated checkpoint therapy or ACT and the checkpoint molecule E2F.

### BACKGROUND OF THE INVENTION

Checkpoints are built into the machinery of the cell proliferation cycle to protect chromosomal integrity. The approximately 10¹⁶ cell multiplications that occur during the human life span, together with inevitable errors in DNA replication, and exposure to ultraviolet rays and mutagens, underscores the requirement for accurate checkpoint function. In the simplest model, four major checkpoints monitor the integrity of genetic material. These checkpoints occur during cell-cycle progression, making certain that previous steps have been adequately completed before advancing along the cycle. DNA synthesis begins only past the restriction point (R point), where the cell determines if preparation during G1 has been satisfactory for cell-cycle continuation (Pardee AB. (1974) Proc. Natl. Acad. Sci. U.S.A., 71:1286). The second checkpoint occurs during replicon initiation in S phase. The third checkpoint takes place in the G2 phase, where DNA synthesis is completed and assessed prior to chromosome segregation. The fourth checkpoint occurs in M- phase, termed the mitotic checkpoint. Delays in the cell cycle, made possible by checkpoints, facilitate repair and minimize dangerous replication and segregation of damaged DNA. Cells are generally thought to undergo apoptosis when the DNA damage is irreparable, after they unsuccessfully commit to repair DNA, or when conditions are adverse for their growth.

In order to understand checkpoint regulation, the workings of the cell cycle must be clearly outlined. Briefly, it is the family of CDKs and their partner cyclins, which form the “engine” of the cell cycle (Murray A. and Hunt T., The Cell Cycle; Freeman, N.Y., 1993). Active forms of CDKs are a complex of a kinase and a cyclin. These complexes undergo changes in the kinase and cyclin components, thereby driving the cell from one stage of the cell cycle to the next. A succession of kinase subunits in a specific order, namely CDK4, CDK2, and CDC2, is expressed along with the succession of cyclins D, E, A, and B, as the cells progress from G1 to mitosis (Sherr, C J (1993) Cell 73:1059). CDK4 is complexed with several D cyclins and its function is induced early in the cycle, likely in response to growth factors. CDK2 can be complexed either to cyclin E or A and is essential for DNA replication. CDC2 can be complexed with cyclins A or B and is essential for mitosis. Thus, in a simplified outline, cell-cycle progression is achieved by various proteins activated or inactivated by phosphorylation, as a result of activity of the CDKs during that stage. However, regulation of cell-cycle progression is much more complex; it involves transcription of cyclin genes, degradation of cyclin proteins, modification of CDKs by phosphorylation, and a number of positive and negative feedback loops that contribute to cell-cycle progression (Hartwell L H and Kastan M B. Science 1994, 266:1821-1828).

Checkpoints serve as integral components of cellular physiology. They are more than surveyors of occasional DNA damage. Their multifaceted role in cellular homeostasis involves not only control of cell-cycle progression, but is also an integral part of activation of DNA repair, composition of telomeric chromatin, activation of transcriptional programs, telomere length and induction of apoptosis (Zhou, B-B S. and Elledge, S J. (2000) Nature 408:433-439). In simplest terms, checkpoint regulation of damage control consists of sensing damage, transduction of information regarding state of DNA and ultimately the execution of DNA damage response by effectors.

Although sensors of DNA damage have not yet been identified, much work has been done on transducers of information regarding DNA damage. Ataxia telangiectasia mutated (ATM) gene and ATM-Rad3-related (ATR) gene relay information to a downstream set of transducers composed of checkpoint kinases (CHK), the Chk1 and Chk2. Ultimate effectors of this cascade are the substrates of Chk1 and Chk2, which are directly involved in DNA repair and transcriptional regulation, namely BRCA1, p53 and Cdc25C. This network, composed of sensors, transducers and effectors is essentially the workhorse of checkpoint execution, which regulates cell-cycle progression.

ATM and ATR, protein kinases related to the intracellular signaling molecule phosphatidylinositol 3-kinase (PI 3-kinase), thus far have been identified as the most proximal transducers of DNA damage (Jackson, S P. (1997) Int. J. Biochem. Cell Bio 29:935; Elledge, S J (1996) Science 274:1664-1672). Defective ATM was identified in patients with ataxia telangiectasia, a disorder that includes increased incidence of cancer in addition to other features. Today, it is believed that ATM responds to IR damage, whereas ATR primarily controls cellular response to other types of damage such as UV or hydroxyurea (Zhou, B-BS and Elledge, S J. (2000) Nature 408:433-439). Moreover, it was shown that ATM is needed for G1 arrest (Kastan M B, et al. (1992) Cell 71:587-597), reduction of DNA synthesis (Painter, R B and Young, B R (1980) Proc Natl Acad Sci USA 77:7315-7317) and G2 arrest (Paules R S, et al. (1995) Cancer Res 55:1763-1773) in response to IR. In addition, ATR was shown to play a role in the G2/M checkpoint response following X-irradiation (Wright J A et al. (1998) Proc. Natl Acad. Sci. USA 95:7445-7450).

The exact pathways of how ATM and ATR are able to transduce information on DNA damage are not yet fully defined. However, some of the substrates on which ATM and ATR act have been identified. Chk1 and Chk2, serine/threonine kinases, were shown to be substrates for ATR and ATM, respectively. Chk1 is significantly phosphorylated in response to hydroxyurea and UV light, but only moderately phosphorylated in response to IR (Zhou, B-BS and Elledge, S J. (2000) Nature 408:433-439). Moreover, mutant mice lacking either Chk1 or ATR show similar phenotypes, suggesting that ATR acts on Chk1 and that the latter is a key effector in the response pathway to UV and hydroxyurea damage. Unlike Chk1, Chk2 is phosphorylated and activated following IR damage by ATM (Matsuoka S, Huang M and Elledge S J (1998) Science 282:1893-1897). Furthermore, absence of Chk2 prevented UV treated cells from activating p53, a tumor suppressor, and p21, a CDK inhibitor and p53 substrate, thereby abrogating G1 arrest (Hirao A et al. (2000) Science 287:1824-1827). Although it has been shown that both ATM and Chk2 phosphorylate p53, the exact pathway of p53 induction in response to IR damage has not yet been defined (Zhou, B-BS and Elledge, S J. (2000) Nature 408:433-439). In addition, both ATM and ATR have been shown to phosphorylate p53 and BRCA1 both in vitro and in vivo (Zhou, B-BS and Elledge, S J. (2000) Nature 408:433-439), however ATM acts in response to IR, while ATR does so in response to other forms of damage.

It seems that ATM and ATR are able to not only directly affect effector tumor suppressor molecules such as p53/p21 and BRCA1, but they can also pass on information to downstream transducers such as Chk1 and Chk2. For a G1/S arrest, Chk1 and Chk2 can act via p53/p21 and BRCA1, whereas a G2 arrest is achieved through Chk1 or Chk2 maintenance of inhibitory phosphorylation of Cdc2 (Nurse P (1997) Cell 91:865-867). More specifically, in response to DNA damage, Chk1 or Chk2 phosphorylate Cdc25, a dual specificity phosphatase for Cdc2. The phosphorylated form of Cdc25 consequently translocates into the cytoplasm from the nucleus becoming Cdc25C, where it then retained following binding to 14-3-3 proteins (Peng C-Y, et al. (1997) Science 277:1501-1505; Dalal S N, et al. (1999) Mol. Cell Biol. 19:4465-4479). 14-3-3 proteins, 7 in total, are highly conserved, phosphoserine-binding proteins involved in cellular proliferation, checkpoint control and apoptosis (Aitken A. (1996) Trends Cell Biol 6:341-347). When 14-3-3[sigma] binds Cdc25C in the cytoplasm, the latter is unable to translocate into the nucleus to dephosphorylate and thereby activate Cdc2, a Cdk responsible for G2/M progression, effectively causing G2/M arrest. To complicate this picture further, p53, a G1/S regulator, also affects G2/M arrest maintenance since it induces expression of 14-3-3[sigma] (Hermeking H. et al. (1997) Mol. Cell 1:3-13).

The connection between checkpoint activation and cell death is poorly understood. More specifically, it remains unknown how checkpoint activation leads to cell death. It seems that there are at least three checkpoint-dependent pro-apoptotic conditions that occur in a cancer cell. The first condition is dependent on activation of a checkpoint in the presence of DNA damage. Current anti-cancer drugs and X-rays induce cancer cell death by creating DNA damage. Damaged DNA activates checkpoints, where cells may commit to apoptosis if DNA damage is irreparable. Supporting evidence for this mechanism is that mutations in the checkpoint molecule p53 lead to resistance to apoptosis induced by X-irradiation and DNA damaging drugs. Paradoxically, these therapeutic modalities show modest selectivity against cancer in vivo. So what accounts for the selectivity? One possibility is that mutations in the p53 pathway lead to two separate effects on cell death: resistance to apoptosis because of checkpoint defects and promotion of apoptosis because of defective coordination of checkpoints. According to this idea, the overall sensitivity of cancer cells to apoptosis will depend on which one dominates. The presence of mutations in other molecules in the checkpoint network may determine the balance.

The second pro-apoptotic condition that can occur in cancer cells has been exploited for enhancing chemotherapy or radiation therapy. In theory, further inhibiting the already weakened checkpoint control should promote accumulation of DNA damage, which will eventually result in cell death because of a catastrophic amount of DNA damage. For example, most cancer cells harbor defects in the G1 checkpoint. Abrogation of the G2 checkpoint by caffeine promotes cell death in cells with DNA damage.

The third pro-apoptotic condition can be induced by activation of one or more checkpoints without causing DNA damage. This condition is completely different from the scenario under the first condition where activation of a checkpoint is secondary to DNA damage. Under this third condition, cell death is likely to occur because of endogenous DNA damage accumulated in cancer cells as well as “collisions” between the proliferation drive of cancer cells and the activated checkpoint “brakes”.

Support for this “collision” model was an experiment with c-myc. It was observed that cells with over expressed c-myc are more prone to apoptosis in the absence of growth factors. To explain this phenomenon, it was proposed that the activation of cell cycle checkpoints by withdrawing growth factors collided with the proliferation drive caused by c-myc, which resulted in enhanced apoptosis. Similar apoptotic effects have been observed for other oncogenes and for the HIV tat protein.

Cell cycle checkpoints have been attractive targets for cancer chemotherapy. The first reported approach to target checkpoints was to exploit the chemical sensitivity resulting from the loss of checkpoint function. Since cells arrest in G2/M after treatment with DNA-damaging agents, such as chemotherapeutic agents and X-rays, the therapeutic approach was devised to eliminate the G2/M delay caused by DNA damaging agents, thereby creating lethal mitosis of cancer cells, a property first observed with caffeine and its analogs. Several caffeine analogs have been discovered with potential for cancer therapy.

Many disease conditions are affected by the development of poorly regulated cell cycle checkpoint controls and a defective apoptotic response. For example, neoplasias may result, at least in part, when cell proliferation signals inappropriately exceed cell death signals. Furthermore, some DNA viruses such as Epstein-Barr virus, African swine fever virus and adenovirus, parasitize the host cellular machinery to drive their own replication and at the same time modulate apoptosis to repress cell death and allow the target cell to reproduce the virus. Moreover, certain disease conditions such as cancer including drug resistant cancer, lymphoproliferative conditions, arthritis, inflammation, autoimmune diseases, immunodeficiency diseases, including AIDS, senescence, neurodegenerative diseases, ischemia and reperfusion, infertility, wound-healing and the like may result from a defect in cell cycle checkpoint control and cell death regulation. In such disease conditions, it would be desirable to regulate checkpoint activation and apoptotic mechanisms.

Since there is an unmet need in regard to checkpoint and cell cycle regulation, it is desirable to identify therapeutic agents that do not damage DNA and do not stabilize microtubules; that modulate checkpoint control and to utilize these agents for the simultaneous and transient activation of checkpoints to induce synergistic and selective apoptosis. This method can be used as a basis for treatment modalities and the discovery of new drugs for advantageously modulating cell cycle progression and checkpoint control in disease conditions that involve inappropriate repression of apoptosis.

### SUMMARY OF THE INVENTION

The present invention is based on the transient activation of cell cycle checkpoints. More specifically, the present invention discloses methods of selectively modulating the activation of early cell cycle checkpoints (e.g. G1 and S), which are commonly defective in cancer cells, without substantial DNA damage and without substantial microtubule stabilization, thereby inducing apoptosis in cancer cells without affecting normal cells. The activation of the early cell cycle checkpoints and the induction of apoptosis by these compounds appears to be caused by selective upregulation of members of the E2F family of transcription factors (including but not limited to E2F-1, E2F-2, E2F-3) in cancer cells vs. normal cells.

In one embodiment, the present invention relates to a method of treating cancer by administering a cell cycle checkpoint activation modulator to a subject in need thereof, wherein the modulator: does not damage DNA and preferably does not stabilize microtubules and is administered in a dosage effective manner to treat cancer in the subject, wherein the modulator is not β-lapachone. Preferably the checkpoint modulated is commonly defective in cancer cells (i.e. G1, S, G2, M).

In another embodiment, the present invention relates to a method of treating cancer by administering a cell cycle checkpoint activation modulator to a subject in need thereof, wherein the modulator: does not damage DNA and preferably does not stabilize microtubules; is administered in a dosage effective manner to treat cancer in the subject; and elevates the level of a member of the E2F family of transcription factors (including but not limited to E2F-1, E2F-2 or E2F-3), wherein the modulator is not β-lapachone. Preferably the activation of the checkpoint is accompanied by an elevation of a member of the E2F family of transcription factors.

In another embodiment, the present invention relates to a method of treating cancer by administering a cell cycle checkpoint activation modulator to a subject in need thereof, wherein the modulator: does not damage DNA and preferably does not stabilize microtubules; is administered in a dosage effective manner to treat cancer in the subject; and elevates the level of the transcription factor E2F-1, wherein the modulator is not β-lapachone. Preferably the activation of the checkpoint is accompanied by an elevation of the transcription factor E2F-1.

The cell cycle checkpoint activation modulator can inhibit cellular proliferation or induce apoptosis. As used herein, a “modulator” is a molecule which stimulates (i.e. induces) or inhibits cell cycle checkpoint activation. The cell cycle checkpoint activation modulator can be a G1 or S phase checkpoint modulator, or a G1 and S phase checkpoint modulator, a non-peptide or non-protein and can have a molecular weight of less than 5 kD. In preferred embodiments, the cell cycle checkpoint activation modulator can be 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione or 3,4-dihydro-4,4-dimethyl-2H-naphtho[1,2-b]thiopyran-5,6-dione.

The subject can be a human and the cell cycle checkpoint activation modulator can be administered parenterally, intravenously, orally or topically. In another embodiment, the effective dosage is not cytotoxic to non-cancerous (e.g. normal) cells and does not affect the viability of non-cancerous cells.

The cell cycle checkpoint activation modulator can be administered in combination with a chemotherapeutic agent. The chemotherapeutic agent can be a microtubule targeting drug, a topoisomerase poison drug or a cytidine analogue drug. In preferred embodiments, the chemotherapeutic agent can be Taxol® (paclitaxel), lovastatin, minosine, tamoxifen, gemcitabine, araC, 5-fluorouracil (5-FU), methotrexate (MTX), docetaxel, vincristin, vinblastin, nocodazole, teniposide, etoposide, adriamycin, epothilone, navelbine, camptothecin, daunonibicin, dactinomycin, mitoxantrone, amsacrine, epirubicin or idarubicin.

In another embodiment, the present invention relates to a method for treating or preventing an apoptosis-associated disorder by administering a cell cycle checkpoint activation modulator to subject in need thereof, wherein the modulator: does not damage DNA and does not stabilize microtubules; and is administered in a therapeutically effective amount to induce apoptosis in the subject, wherein the modulator is not β-lapachone, thereby treating or preventing an apoptosis-associated disorder.

In another embodiment, the present invention relates to a method of inducing apoptosis in a subject by administering a cell cycle checkpoint activation modulator to subject in need thereof, wherein the modulator: does not damage DNA and does not stabilize microtubules; and is administered in a therapeutically effective amount to induce apoptosis in the subject, wherein the modulator is not β-lapachone, thereby inducing apoptosis is the subject.

In another embodiment, the present invention relates to a method of inducing apoptosis in a cell by contacting the cell with a cell cycle checkpoint activation modulator, wherein the modulator: does not damage DNA and does not stabilize microtubules; and is in a dosage effective to induce apoptosis in the cell, wherein the modulator is not β-lapachone, thereby inducing apoptosis in the cell.

In another embodiment, the present invention relates to a method for screening for a cell cycle checkpoint activation modulator by contacting a cancer cell with a candidate compound, and measuring the degree (or extent) of elevation of a member of the E2F family of transcription factors (including but not limited to E2F-1, E2F-2 or E2F-3), if present, where an increase in E2F in the presence of the compound, as compared to the absence of the compound, indicates that the compound is an inducer of apoptosis.

In another embodiment, the present invention relates to a method for screening for a cell cycle checkpoint activation modulator by contacting a cancer cell with a candidate compound, and measuring the degree (or extent) of elevation of the transcription factor E2F-1, if present, where an increase in E2F-1 in the presence of the compound, as compared to the absence of the compound, indicates that the compound is an inducer of apoptosis.

In another embodiment, the present invention relates to a method for screening for a cell cycle checkpoint activation modulator by contacting a cell with a candidate compound, and measuring the degree (or extent) of apoptosis, if present, where an increase in apoptosis in the presence of the compound, as compared to the absence of the compound, indicates that the compound is an inducer of apoptosis.

In preferred embodiments, the screening methods identify cell cycle checkpoint activation modulators. In additional preferred embodiments, the present invention relates to a method of treating cancer by administering a cell cycle checkpoint activation modulator identified by the screening methods, to a subject in need thereof, where the cell cycle checkpoint activation modulator treats the cancer.

In another embodiment, the present invention relates to a method for screening for a compound effective for treating cancer by contacting a cancer cell with a candidate compound, and measuring the degree (or extent) of elevation of a member of the E2F family of transcription factors (i.e. E2F-1, E2F-2 or E2F-3), if present, where an increase in E2F in the presence of the compound, as compared to the absence of the compound, indicates that the compound is an inducer of apoptosis.

In another embodiment, the present invention relates to a method for screening for a compound effective for treating cancer by contacting a cancer cell with a candidate compound, and measuring the degree (or extent) of elevation of the transcription factor E2F-1, if present, where an increase in E2F-1 in the presence of the compound, as compared to the absence of the compound, indicates that the compound is an inducer of apoptosis.

In another embodiment, the present invention relates to a method for screening for a compound effective for treating cancer by contacting a cell with a candidate compound, and measuring the degree (or extent) of apoptosis, if present, where an increase in apoptosis in the presence of the compound, as compared to the absence of the compound, indicates that the compound is an inducer of apoptosis.

In preferred embodiments, the screening methods identify compounds effective for treating cancer. In additional preferred embodiments, the present invention relates to a method of treating cancer by administering a compound effective for treating cancer identified by the screening methods, to a subject in need thereof, where the compound effective for treating cancer treats the cancer.

Other features and advantages of the invention will be apparent from the following detailed description and claims.

### BRIEF DESCRIPTION OF THE DRAWINGS

FIG. 1 is a schematic representation of the cell cycle showing which checkpoints are affected by β-Lapachone and Taxol® and the effects of β-Lapachone and Taxol® on cancer cell survival.

FIG. 2 shows the differential effects of β-Lapachone on human multiple myeloma (MM) cells vs. normal human Peripheral Blood Mononuclear Cells (PBMC).

FIG. 3 is a photograph of a colony formation assay showing the differential effects of β-Lapachone on human breast cancer cells (MCF-7) vs. normal human breast epithelial cells (MCF-10A).

FIG. 4 is a photograph of an apoptosis assay and corresponding bar graph of an MTT Assay showing β-Lapachone induced apoptosis in human colon carcinoma cells (DLD1).

FIG. 5 is a photograph of a histogram showing that β-Lapachone induces apoptosis in human colon carcinoma cells (DLD1 and SW480) as demonstrated by the appearance of a sub-G1 fraction, whereas no apoptosis is seen in normal human colon cells (NCM460).

FIG. 6 is a photograph of a Western blot showing β-Lapachone stress induces cytochrome c release and PARP cleavage, both evidence of apoptosis.

FIG. 7 is a photograph of a gel mobility shift assay showing the binding of nuclear proteins from β-Lapachone-treated and -untreated human colon carcinoma cells (DLD1) and normal colon cells (NCM460).

FIG. 8 is a photograph of a Western blot showing that E2F-1 protein expression is upregulated by β-Lapachone in human pancreatic cancer cells (Paca-2).

FIG. 9 is a photograph of a Western blot showing that E2F-1 protein and closely related family members E2F-2 and E2F-3 protein expression is upregulated by β-Lapachone in human colon cancer cells (SW480)

FIG. 10 is a bar graph showing β-Lapachone induced elevation of E2F-1 levels.

FIG. 11 is a photograph of a Western blot showing β-Lapachone induced elevation of E2F-1 levels in human colon cancer cells (SW480) and normal colon cells (NCM460).

FIG. 12 is a bar graph showing the cytotoxic effects of β-Lapachone in combination with GL331 in human prostate cancer cells (PC-3).

FIG. 13 is a bar graph showing the cytotoxic effects of β-Lapachone in combination with gemcitabine in human pancreatic cancer cells (Paca-2).

### DETAILED DESCRIPTION OF THE INVENTION

The present invention is based in part on methods for the transient activation of checkpoints, called Activated Checkpoint Therapy™, or ACT. Briefly, cancer cells are defective in their checkpoint functions secondary to mutations in one of their molecular modulators, e.g. p53. It is in part for this reason that cancer cells have accumulated genetic errors during the carcinogenic process. Therapeutic agents that transiently activate checkpoint function can selectively promote cell death in cancer cells, since apoptosis appears to be induced by the conflict between the uncontrolled-proliferation drive in cancer cells and the checkpoint delays induced artificially. The ACT method takes advantage of the tendency of apoptosis to occur at checkpoints during the cell proliferation cycle by transiently activating one or more checkpoints, thereby producing conflicting signals regarding cell cycle progression vs. arrest. If more than one checkpoint is activated, cancer cells with uncontrolled proliferation signals and genetic abnormalities are blocked at multiple checkpoints, creating “collisions” that promote synergistic apoptosis.

The ACT method offers selectivity against cancer cells as compared to normal cells and is therefore safer than less selective therapies. Firstly, the ACT method transiently activates but does not disrupt the checkpoints. Activation of checkpoints in the absence of DNA damage, microtubule stabilization and oncogene activation simply mimics a physiological response and thus does not trigger cell death. Secondly, normal cells with well-controlled proliferation signals can be delayed at these checkpoints in a regulated fashion, resulting in no apoptosis-prone collisions. Thirdly, normal cells with intact G1 checkpoint control are expected to arrest in G1. Cancer cells, on the other hand, are expected to be delayed in S-, G2-, and M-phases, since most cancer cells harbor G1 checkpoint defects, making cancer cells more sensitive to drugs imposing S and M phase checkpoints.

Cell Cycle Checkpoint Activation Modulators

Two compounds that are known to modulate checkpoint activation without substantial DNA damage are β-Lapachone and Taxol®. More importantly as described herein, several compounds, including but not limited to: 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione and 3,4-dihydro-4,4-dimethyl-2H-naphtho[1,2-b]thiopyran-5,6-dione and β-Lapachone modulate checkpoint activation without substantial DNA damage and without substantial microtubule stabilization. Compounds which modulate checkpoint activation without substantial DNA damage and without substantial microtubule stabilization are critical for inducing cell death (i.e. apoptosis) in cancer cells without affecting normal cells.

Damage to cellular DNA, can be caused by radiation or by most conventional chemotherapeutic agents, including but not limited to alkylating agents (e.g. cyclophosphamide), platinum analogues and topoisomerase poisons (e.g. the anthracyclines and campothecins), includes DNA lesions (e.g. strand breaks, cross-linking, alkylation, adduct formation, or stabilization of the topisomerase/DNA cleavable complex), which can result in suspension of progress through the cell cycle while the cell attempts to repair the detected damage. Microtubule stabilization can be the prevention of microtubule assembly (i.e. by the Vinca alkyloids) or depolymerization (i.e. by the taxanes), possibly through binding of chemotherapeutic agents to sites on the tubulin subunits of the microtubule, possibly inducing metaphase arrest in dividing cells (cyclophosphamide).

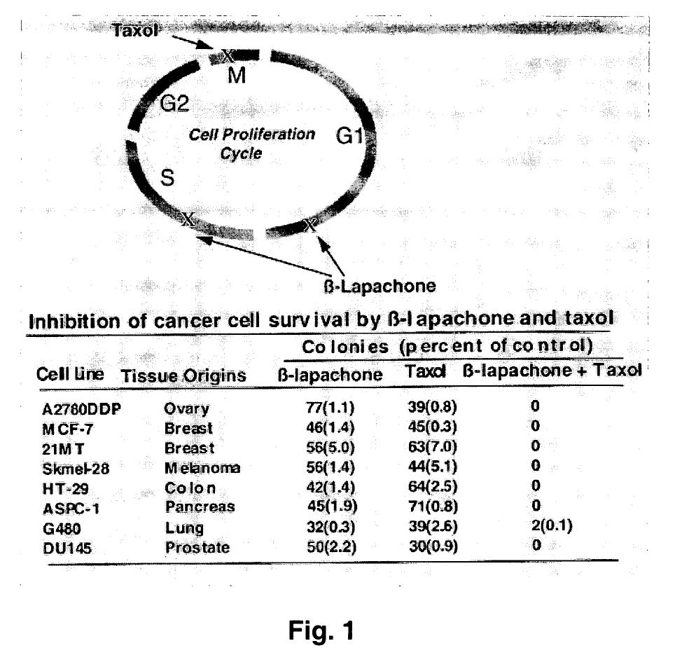

These compounds function at different checkpoints in the cell cycle. While Taxol® activates the mitotic checkpoint, β-Lapachone, 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione and 3,4-dihydro-4,4-dimethyl-2H-naphtho[1,2-b]thiopyran-5,6-dione induce G1 plus S-phase checkpoint delays (FIG. 1). The combination of β-Lapachone, 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione or 3,4-dihydro-4,4-dimethyl-2H-naphtho[1,2-b]thiopyran-5,6-dione with Taxol® causes simultaneous cell cycle checkpoint delays at the G1/S and G2/M transitions, resulting in synergistic apoptotic activity against a wide spectrum of human cancer cells in vitro (FIG. 1). In the presence of β-Lapachone, the effective Taxol® concentration was reduced by at least 10 fold. More importantly, this combination has been shown to have unusually potent activity without toxicity in xenografted human tumors in animal models (U.S. Publication No. US-2002-0169135-A1). The ACT method can be utilized similarly to treat patients with solid malignancies in a variety of tissues.

β-Lapachone (3,4-dihydro-2,2-dimethyl-2H-naphtho [1,2-b] pyran-5,6-dione), a simple non-water soluble orthonapthoquinone, was first isolated in 1882 by Paterno from the heartwood of the lapacho tree (See Hooker, S C, (1936) I. Am. Chem. Soc. 58:1181-1190; Goncalves de Lima, O, et al., (1962) Rev. Inst. Antibiot. Univ. Recife. 4:3-17). The structure of β-Lapachone was established by Hooker in 1896 and it was first synthesized by Fieser in 1927 (Hooker, S C, (1936) I. Am. Chem. Soc. 58:1181-1190). β-Lapachone can be obtained by simple sulfuric acid treatment of the naturally occurring lapachol, which is readily isolated from Tabebuia avellenedae growing mainly in Brazil, or is easily synthesized from seeds of lomatia growing in Australia (Li, C J, et al., (1993) J. Biol. Chem. 268:22463-33464).

β-Lapachone has been shown to have a variety of pharmacological effects. The present inventors have demonstrated that β-Lapachone inhibits viral replication and gene expression directed by the long terminal repeat (LTR) of the human immunodeficiency virus type I (Li, C J et al., (1993) Proc. Natl. Acad. Sci. USA 90:1839-1842). β-Lapachone was investigated as a novel and potent DNA repair inhibitor that sensitizes cells to ionizing radiation and DNA damaging agents (Boorstein, R J et al., (1984) Biochem Biophys. Res. Commun. 118:828-834; Boothman, et al., (1989) Cancer Res. 49:605-612). The present inventors have reported that β-Lapachone and its derivatives inhibit eukaryotic topoisomerase I through a different mechanism than does camptothecin, which may be mediated by a direct interaction of β-Lapachone with topoisomerase I rather than stabilization of the cleavable complex (Li, C J et al., (1999) J. Biol. Chem. 268:22463-22468). The present inventors and others have reported that β-Lapachone induces cell death in human prostate cancer cells (See Li, C J et al., 1 (1995) Cancer Res. 55:3712-3715). Furthermore, the present inventors found that β-Lapachone induces necrosis in human breast cancer cells, and apoptosis in ovary, colon, and pancreatic cancer cells through induction of caspase (Li, Y Z et al., (1999) Molecular Medicine 5:232-239). Methods for formulating β-Lapachone or its derivatives or analogs can be accomplished as described in U.S. Pat. No. 6,458,974 and U.S. Publication No. US-2003-0091639-A1.

Methods of Modulating Checkpoint Activation and Treating Cancer

A variety of methods are currently available for inducing cell death in cancer cells. However, they all suffer the problem of selectivity as they affect cancer cells and normal cells equally. The present invention is directed to a method to selectively modulate (i.e. stimulate or inhibit) checkpoint activation and promote apoptosis in cancer cells. In one aspect, stimulation of unscheduled expression of a checkpoint molecule, e.g. E2F, via a non-DNA damaging, non-microtubule stabilizing molecule selectively triggers cell death in cells with defective checkpoints, a hallmark of cancer and pre-cancer cells. As used herein, “E2F” is the E2F transcription factor family (including but not limited to E2F-1, E2F-2, E2F-3). The claimed method does not induce cell death in normal cells with their intact checkpoint control.

Several compounds, including but not limited to: 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione and 3,4-dihydro-4,4-dimethyl-2H-naphtho[1,2-b]thiopyran-5,6-dione and β-Lapachone, induce unscheduled expression of checkpoint molecules, e.g. E2F, independent of substantial DNA damage, microtubule stabilization and cell cycle stages. In normal cells with their intact regulatory mechanisms, such an imposed expression of a checkpoint molecule results in a transient expression pattern and causes no substantial consequence. In contrast, cancer and pre-cancer cells have defective mechanisms, which result in unchecked and persistent expression of unscheduled checkpoint molecules, e.g. E2F, leading to selective cell death in cancer and pre-cancer cells.

In one embodiment, the present invention relates to a method of treating cancer by administering a cell cycle checkpoint activation modulator to a subject in need thereof, wherein the modulator: does not damage DNA and preferably does not stabilize microtubules; is administered in a dosage effective manner to treat cancer in the subject, wherein the modulator is not β-lapachone. Preferably the checkpoint modulated is commonly defective in cancer cells (i.e. G1, S, G2, M).

In another embodiment, the present invention relates to a method of treating cancer by administering a cell cycle checkpoint activation modulator to a subject in need thereof, wherein the modulator: does not damage DNA and preferably does not stabilize microtubules; is administered in a dosage effective manner to treat cancer in the subject; and elevates (i.e. induces) the level of a member of the E2F family of transcription factors (including but not limited to E2F-1, E2F-2 or E2F-3), wherein the modulator is not β-lapachone. Preferably the activation of the checkpoint is accompanied by an elevation of a member of the E2F family of transcription factors.

In another embodiment, the present invention relates to a method of treating cancer by administering a cell cycle checkpoint activation modulator to a subject in need thereof, wherein the modulator: does not damage DNA and preferably does not stabilize microtubules; is administered in a dosage effective manner to treat cancer in the subject; and elevates (i.e. induces) the level of the transcription factor E2F-1, wherein the modulator is not β-lapachone. Preferably the activation of the checkpoint is accompanied by an elevation of the transcription factor E2F-1.

The stimulation of unscheduled expression of checkpoint molecules can be achieved via genetic methods, protein or peptides, and small molecules that can be utilized for the treatment and prevention of various cancers and cell proliferative disorders. As used herein, “cell proliferative disorder” refers to conditions in which the unregulated and/or abnormal growth of cells can lead to the development of an unwanted condition or disease, which can be cancerous or non-cancerous.

In additional embodiments, the cell cycle checkpoint activation modulator used to treat cancer can inhibit cellular proliferation or induce apoptosis. The cell cycle checkpoint activation modulator can be a G1 or S phase checkpoint modulator, or a G1 and S phase checkpoint modulator. In another embodiment, the cell cycle checkpoint activation modulator can be a G2 checkpoint modulator. The cell cycle checkpoint activation modulator can be a non-peptide or non-protein and preferably can have a molecular weight of less than 5 kD.

In a preferred embodiment, the present invention relates to a method of treating or preventing cancer by administering a cell cycle checkpoint activation modulator to a subject in need thereof, where administration of the cell cycle checkpoint activation modulator results in one or more of the following: accumulation of cells in G1 and/or S phase of the cell cycle, cytotoxicity via apoptosis in cancer cells but not in normal cells, antitumor activity in animals with a therapeutic index of at least 2, and modulation of cell cycle checkpoint activation (i.e. elevation of a member of the E2F family of transcription factors). As used herein, “therapeutic index” is the maximum tolerated dose divided by the efficacious dose.

In more preferred embodiments, the cell cycle checkpoint activation modulator can be 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione or 3,4-dihydro-4,4-dimethyl-2H-naphtho[1,2-b]thiopyran-5,6-dione.

In additional embodiments, the subject can be any mammal, e.g., a human, a primate, mouse, rat, dog, cat, cow, horse, pig. In another embodiment, the subject can be any non-mammal, e.g., a reptile, bird. In various embodiments, the subject is susceptible to cancer, cell proliferative disorder, an autoimmune disorder or disorder of the like. The cell cycle checkpoint activation modulator can be administered parenterally, intravenously, orally or topically. In preferred embodiments, the effective dosage is not cytotoxic to non-cancerous (i.e. normal) cells and does not affect the viability of non-cancerous cells.

In additional embodiments, the cell cycle checkpoint activation modulator can be administered in combination with a chemotherapeutic agent. The chemotherapeutic agent can be a microtubule targeting drug, a topoisomerase poison drug or a cytidine analogue drug. In preferred embodiments, the chemotherapeutic agent can be Taxol® (paclitaxel), lovastatin, minosine, tamoxifen, gemcitabine, araC, 5-fluorouracil (5-FU), methotrexate (MTX), docetaxel, vincristin, vinblastin, nocodazole, teniposide, etoposide, adriamycin, epothilone, navelbine, camptothecin, daunonibicin, dactinomycin, mitoxantrone, amsacrine, epirubicin or idarubicin.

In another embodiment, the present invention relates to a method of treating cancer by administering a compound to a subject in need thereof, wherein the compound: is administered in a dosage effective manner to treat cancer in the subject; and elevates (i.e. induces) the level of a member of the E2F family of transcription factors (including but not limited to E2F-1, E2F-2 or E2F-3), wherein the compound is not β-lapachone. Preferably the compound is administered in combination with a chemotherapeutic agent.

The chemotherapeutic agent can be a microtubule targeting drug, a topoisomerase poison drug or a cytidine analogue drug. In preferred embodiments, the chemotherapeutic agent can be Taxol® (paclitaxel), lovastatin, minosine, tamoxifen, gemcitabine, araC, 5-fluorouracil (5-FU), methotrexate (MTX), docetaxel, vincristin, vinblastin, nocodazole, teniposide, etoposide, adriamycin, epothilone, navelbine, camptothecin, daunonibicin, dactinomycin, mitoxantrone, amsacrine, epirubicin or idarubicin.

Methods of Modulating Checkpoint Activation and Inducing Apoptosis

Also included in the invention are methods of modulating cell cycle checkpoint activation, inducing apoptosis and treating or preventing an apoptosis-associated disorder. In one embodiment, the present invention relates to a method for treating or preventing an apoptosis-associated disorder by administering a cell cycle checkpoint activation modulator to subject in need thereof, wherein the modulator: does not damage DNA and preferably does not stabilize microtubules; and is administered in a therapeutically effective amount to induce apoptosis in the subject, wherein the modulator is not β-lapachone, thereby treating or preventing an apoptosis-associated disorder.

In another embodiment, the present invention relates to a method of inducing apoptosis in a subject by administering a cell cycle checkpoint activation modulator to subject in need thereof, wherein the modulator: does not damage DNA and preferably does not stabilize microtubules; and is administered in a therapeutically effective amount to induce apoptosis in the subject, wherein the modulator is not β-lapachone, thereby inducing apoptosis in the subject.

In another embodiment, the present invention relates to a method of inducing apoptosis in a cell by contacting the cell with a cell cycle checkpoint activation modulator, wherein the modulator: does not damage DNA and preferably does not stabilize microtubules; and is in a dosage effective to induce apoptosis in the cell, wherein the modulator is not β-lapachone, thereby inducing apoptosis in the cell. The cell population that is exposed to, i.e., contacted with, a cell cycle checkpoint activation modulator can be any number of cells, i.e., one or more cells, and can be provided in vitro, in vivo, or ex vivo. The cell population can be eukaryotic or prokaryotic cells.

In additional embodiments, cell cycle checkpoint activation modulator can be a G1 or S phase checkpoint modulator, or a G1 and S phase checkpoint modulator. In another embodiment, the cell cycle checkpoint activation modulator can be a G2 phase checkpoint modulator. The cell cycle checkpoint activation modulator can be a non-peptide or non-protein and preferably can have a molecular weight of less than 5 kD.

In a preferred embodiment, the present invention relates to a method of treating or preventing an apoptosis-associated disorder or a method of inducing apoptosis by administering a cell cycle checkpoint activation modulator to a subject in need thereof or by contacting a cell with a cell cycle checkpoint activation modulator, where administration/contact of the cell cycle checkpoint activation modulator results in one or more of the following: accumulation of cells in G1 and/or S phase of the cell cycle, cytotoxicity via apoptosis in cancer cells but not in normal cells, antitumor activity in animals with a therapeutic index of at least 2, and modulation of cell cycle checkpoint activation (including but not limited to the elevation of a member of the E2F family of transcription factors).

In more preferred embodiments, the cell cycle checkpoint activation modulator can be 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione or 3,4-dihydro-4,4-dimethyl-2H-naphtho[1,2-b]thiopyran-5,6-dione.

In additional embodiments, the subject can be any mammal, e.g., a human, a primate, mouse, rat, dog, cat, cow, horse, pig. In another embodiment, the subject can be any non-mammal, e.g., a reptile, bird. In various embodiments, the subject is susceptible to cancer, cell proliferative disorder, an autoimmune disorder or disorder of the like. The cell cycle checkpoint activation modulator can be administered parenterally, intravenously, orally or topically. In preferred embodiments, the effective dosage is not cytotoxic to non-cancerous (i.e. normal) cells and does not affect the viability of non-cancerous cells.

In additional embodiments, the cell cycle checkpoint activation modulator can be administered in combination with a chemotherapeutic agent. The chemotherapeutic agent can be a microtubule targeting drug, a topoisomerase poison drug or a cytidine analogue drug. In preferred embodiments, the chemotherapeutic agent can be Taxol® (paclitaxel), lovastatin, minosine, tamoxifen, gemcitabine, araC, 5-fluorouracil (5-FU), methotrexate (MTX), docetaxel, vincristin, vinblastin, nocodazole, teniposide, etoposide, adriamycin, epothilone, navelbine, camptothecin, daunonibicin, dactinomycin, mitoxantrone, amsacrine, epirubicin or idarubicin.

In another embodiment, the present invention relates to a method of treating or preventing an apoptosis-associated disorder or inducing apoptosis by administering a compound to a subject in need thereof, wherein the compound: is administered in a dosage effective manner to treat or prevent an apoptosis-associated disorder or induce apoptosis in the subject; and elevates (i.e. induces) the level of a member of the E2F family of transcription factors (including but not limited to E2F-1, E2F-2 or E2F-3), wherein the compound is not β-lapachone. Preferably the compound is administered in combination with a chemotherapeutic agent.

Some disease conditions are related to the development of a defective down-regulation of apoptosis in the affected cells. For example, neoplasias result, at least in part, from an apoptosis-resistant state in which cell proliferation signals inappropriately exceed cell death signals. Furthermore, some DNA viruses such as Epstein-Barr virus, African swine fever virus and adenovirus, parasitize the host cellular machinery to drive their own replication. At the same time, they modulate apoptosis to repress cell death and allow the target cell to reproduce the virus. Moreover, certain disease conditions such as cancer including drug resistant cancer, cell proliferation disorders, lymphoproliferative conditions, arthritis, inflammation, autoimmune diseases and the like may result from a down regulation of cell death regulation. In such disease conditions, it would be desirable to induce checkpoint activation and promote apoptotic mechanisms as described supra.

Methods for Screening for Cell Cycle Checkpoint Activation Modulators

The invention provides a method (also referred to herein as a “screening assay”) for identifying cell cycle checkpoint activation modulators, i.e., candidate or test compounds or agents (e.g., small molecules, large molecules, peptides, peptidomimetics or other drugs).

In one embodiment, the present invention relates to a method for screening for a cell cycle checkpoint activation modulator by contacting a cancer cell with a candidate compound, and measuring the degree (or extent) of elevation of a member of the E2F family of transcription factors (including but not limited to E2F-1, E2F-2 or E2F-3), if present, where an increase in E2F in the presence of the compound, as compared to the absence of the compound, indicates that the compound is an inducer of apoptosis.

In another embodiment, the present invention relates to a method for screening for a cell cycle checkpoint activation modulator by contacting a cancer cell with a candidate compound, and measuring the degree (or extent) of elevation of the transcription factor E2F-1, if present, where an increase in E2F-1 in the presence of the compound, as compared to the absence of the compound, indicates that the compound is an inducer of apoptosis.

In another embodiment, the present invention relates to a method for screening for a cell cycle checkpoint activation modulator by contacting a cell with a candidate compound, and measuring the degree (or extent) of apoptosis, if present, where an increase in apoptosis in the presence of the compound, as compared to the absence of the compound, indicates that the compound is an inducer of apoptosis.

In preferred embodiments, the present invention also includes cell cycle checkpoint activation modulators (i.e. molecules, compounds, compositions) identified in the screening assays described herein. In additional preferred embodiments, the present invention relates to a method of treating cancer, method of treating or preventing an apoptosis-associated disorder or inducing apoptosis by administering a cell cycle checkpoint activation modulator identified by the screening methods, to a subject in need thereof, where the cell cycle checkpoint activation modulator treats the cancer, treats or prevents the apoptosis-associated disorder or induces apoptosis.

In another embodiment, the present invention relates to a method for screening for a compound effective for treating cancer by contacting a cancer cell with a candidate compound, and measuring the degree (or extent) of elevation of a member of the E2F family of transcription factors (i.e. E2F-1, E2F-2 or E2F-3), if present, where an increase in E2F in the presence of the compound, as compared to the absence of the compound, indicates that the compound is an inducer of apoptosis.

In another embodiment, the present invention relates to a method for screening for a compound effective for treating cancer by contacting a cancer cell with a candidate compound, and measuring the degree (or extent) of elevation of the transcription factor E2F-1, if present, where an increase in E2F-1 in the presence of the compound, as compared to the absence of the compound, indicates that the compound is an inducer of apoptosis.

In another embodiment, the present invention relates to a method for screening for a compound effective for treating cancer by contacting a cell with a candidate compound, and measuring the degree (or extent) of apoptosis, if present, where an increase in apoptosis in the presence of the compound, as compared to the absence of the compound, indicates that the compound is an inducer of apoptosis.

In preferred embodiments, the present invention also includes compounds effective for treating cancer (i.e. molecules, compounds, compositions) identified in the screening assays described herein. In additional preferred embodiments, the present invention relates to a method of treating cancer by administering a compound effective for treating cancer identified by the screening methods, to a subject in need thereof, where the compounds effective for treating cancer treat the cancer.

The cell population that is exposed to, i.e., contacted with, a candidate or test compounds (i.e. a cell cycle checkpoint activation modulator) can be any number of cells, i.e., one or more cells, and can be provided in vitro, in vivo, or ex vivo. The cell population can be eukaryotic or prokaryotic cells.

In a preferred embodiment, the present invention relates to a candidate or test compounds which is identified as a cell cycle checkpoint activation modulator by the screening assays described herein, where administering a cell cycle checkpoint activation modulator to a subject in need thereof or by contacting a cell with a cell cycle checkpoint activation modulator results in one or more of the following: accumulation of cells in G1 and/or S phase of the cell cycle, cytotoxicity via apoptosis in cancer cells but not in normal cells, antitumor activity in animals with a therapeutic index of at least 2, and modulation of cell cycle checkpoint activation (i.e. elevation of a member of the E2F family of transcription factors).

In another embodiment, the cell cycle checkpoint activation modulator identified by the screening assays described herein can be 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione or 3,4-dihydro-4,4-dimethyl-2H-naphtho[1,2-b]thiopyran-5,6-dione.

In another embodiment, the present invention relates to a method for screening for a cell cycle checkpoint activation modulator that binds to cell cycle regulatory proteins, e.g., members of the E2F transcription factor family, or have a modulating (stimulatory or inhibitory) effect on the activity of these proteins, checkpoint activation or the induction of apoptosis.

In another embodiment, the present invention provides a screening assay for detecting anti-cancer agents. In a preferred embodiment, an E2F promoter-reporter construct can be used to screen for anti-cancer drugs. In another embodiment, the present invention provides a method for the development of novel selective drugs for the treatment and prevention of cancers and cell proliferative disorders.

In another embodiment, the invention provides assays for screening candidate or test compounds, which bind to or modulate the activity of cell cycle regulatory proteins or polypeptide or biologically-active portions thereof.

The test compounds of the invention can be obtained using any of the numerous approaches or methods known in the art. In a preferred embodiment, the test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, (1997) Anticancer Drug Design 12: 145.

A “small molecule” as used herein, is meant to refer to a compound that has a molecular weight of less than about 5 kD, more preferably less than about 2 kD and most preferably less than about 1 kD. Small molecules can be, e.g., nucleic acids, peptides, polypeptides, peptidomimetics, carbohydrates, lipids or other organic or inorganic molecules. A “large molecule” as used herein, is meant to refer to a composition that has a molecular weight of greater than about 5 kD. Large molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In another embodiment, an assay is a cell-based assay in which a cell expresses a cell cycle regulatory protein, or a biologically-active portion thereof, and the cell is contacted with a test compound and the ability of the test compound to bind to a cell cycle regulatory protein is determined. The cell, for example, can be of mammalian origin, e.g., human, or a yeast cell. Determining the ability of the test compound to bind to the cell cycle regulatory protein can be accomplished, for example, by coupling the test compound with a radioisotope or enzymatic label such that binding of the test compound to the cell cycle regulatory protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a cell cycle regulatory protein, or a biologically-active portion thereof, with a known compound which binds a cell cycle regulatory protein to form an assay mixture, contacting the assay mixture with a test compound, and determining the ability of the test compound to interact with a cell cycle regulatory protein, wherein determining the ability of the test compound to interact with a cell cycle regulatory protein comprises determining the ability of the test compound to preferentially bind to cell cycle regulatory protein or a biologically-active portion thereof as compared to the known compound.

In another embodiment, an assay is a cell-based assay comprising contacting a cell expressing a cell cycle regulatory protein, or a biologically-active portion thereof, with a test compound and determining the ability of the test compound to modulate (e.g., stimulate or inhibit) the activity of the cell cycle regulatory protein or biologically-active portion thereof. Determining the ability of the test compound to modulate the activity of the cell cycle regulatory protein or a biologically-active portion thereof can be accomplished, for example, by determining the ability of the cell cycle regulatory protein to bind to or interact with a cell cycle regulatory target molecule. As used herein, a “target molecule” is a molecule with which a cell cycle regulatory protein binds or interacts in nature, for example, a molecule on the surface of a cell which expresses a mitochondrial molecule, a cytoplasmic molecule, or a nuclear molecule, a cell cycle regulatory interacting protein, a molecule on the surface of a second cell, a molecule in the extracellular milieu, or a molecule associated with the internal surface of a cell membrane. A cell cycle regulatory target molecule can be a non-cell cycle regulatory molecule or a cell cycle regulatory protein or polypeptide or a large molecule or small molecule of the invention. In one embodiment, a cell cycle regulatory target molecule is a component of a cell cycle pathway that facilitates cellular proliferation as the result of intracellular or extracellular signals. The target, for example, can be a second cell cycle protein that has regulatory activity or a protein that facilitates the progression of the cell cycle.

Determining the ability of the cell cycle regulatory protein to bind to or interact with a cell cycle regulatory target molecule can be accomplished by one of the methods described above for determining direct binding. In one embodiment, determining the ability of the cell cycle regulatory protein to bind to or interact with a cell cycle regulatory target molecule can be accomplished by determining the activity of the target molecule. For example, the activity of the target molecule can be determined by detecting the induction or prevention of apoptosis, detecting induction of a cellular second messenger of the target (i.e. intracellular Ca²⁺, diacylglycerol, IP₃, etc.), detecting catalytic/enzymatic activity of the target using an appropriate substrate, detecting the induction of a reporter gene (comprising a cell cycle regulatory protein-responsive regulatory element operatively linked to a nucleic acid encoding a detectable marker, e.g., luciferase), or detecting a cellular response, for example, cell survival, cellular differentiation, or cell proliferation.

In another embodiment, an assay of the invention is a cell-free assay comprising contacting a cell cycle regulatory protein or biologically-active portion thereof with a test compound and determining the ability of the test compound to bind to the cell cycle regulatory protein or biologically-active portion thereof. Binding of the test compound to the cell cycle regulatory protein can be determined either directly or indirectly as described above. In one such embodiment, the assay comprises contacting the cell cycle regulatory protein or biologically-active portion thereof with a known compound which binds the cell cycle regulatory protein to form an assay mixture, contacting the assay mixture with a test compound, and determining the ability of the test compound to interact with a cell cycle regulatory protein, wherein determining the ability of the test compound to interact with a cell cycle regulatory protein comprises determining the ability of the test compound to preferentially bind to a cell cycle regulatory protein or biologically-active portion thereof as compared to the known compound.

In another embodiment, an assay is a cell-free assay comprising contacting cell cycle regulatory protein or biologically-active portion thereof with a test compound and determining the ability of the test compound to modulate (e.g. stimulate or inhibit) the activity of the cell cycle regulatory protein or biologically-active portion thereof. Determining the ability of the test compound to modulate the activity of a cell cycle regulatory protein can be accomplished, for example, by determining the ability of the cell cycle regulatory protein to bind to a cell cycle regulatory target molecule by one of the methods described above for determining direct binding. In an alternative embodiment, determining the ability of the test compound to modulate the activity of cell cycle regulatory protein can be accomplished by determining the ability of the cell cycle regulatory protein further modulate a cell cycle regulatory target molecule. For example, the catalytic/enzymatic activity of the target molecule on an appropriate substrate can be determined as described, supra.

In another embodiment, the cell-free assay comprises contacting the cell cycle regulatory protein or biologically-active portion thereof with a known compound which binds cell cycle regulatory protein to form an assay mixture, contacting the assay mixture with a test compound, and determining the ability of the test compound to interact with a cell cycle regulatory protein, wherein determining the ability of the test compound to interact with a cell cycle regulatory protein comprises determining the ability of the cell cycle regulatory protein to preferentially bind to or modulate the activity of a cell cycle regulatory target molecule.

In more than one embodiment of the above assay methods of the invention, it may be desirable to immobilize either cell cycle regulatory protein or its target molecule to facilitate separation of complexed from uncomplexed forms of one or both of the proteins, as well as to accommodate automation of the assay. Binding of a test compound to a cell cycle regulatory protein, or interaction of cell cycle regulatory protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-cell cycle regulatory fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or cell cycle regulatory protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of cell cycle regulatory protein binding or activity determined using standard techniques.

Other techniques for immobilizing proteins on matrices can also be used in the screening assays of the invention. For example, either the cell cycle regulatory protein or its target molecule can be immobilized utilizing conjugation of biotin and streptavidin. Biotinylated cell cycle regulatory protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with cell cycle regulatory protein or target molecules, but which do not interfere with binding of the cell cycle regulatory protein to its target molecule, can be derivatized to the wells of the plate, and unbound target or cell cycle regulatory protein trapped in the wells by antibody conjugation. Methods for detecting such complexes, in addition to those described above for the GST-immobilized complexes, include immunodetection of complexes using antibodies reactive with the cell cycle regulatory protein or target molecule, as well as enzyme-linked assays that rely on detecting an enzymatic activity associated with the cell cycle regulatory protein or target molecule.

In another embodiment, modulators of cell cycle regulatory protein expression are identified in a method wherein a cell is contacted with a candidate compound and the expression of cell cycle regulatory mRNA or protein in the cell is determined. The level of expression of cell cycle regulatory mRNA or protein in the presence of the candidate compound is compared to the level of expression of cell cycle regulatory mRNA or protein in the absence of the candidate compound. The candidate compound can then be identified as a modulator of cell cycle regulatory mRNA or protein expression based upon this comparison. For example, when expression of cell cycle regulatory mRNA or protein is greater (i.e., statistically significantly greater) in the presence of the candidate compound than in its absence, the candidate compound is identified as a stimulator of cell cycle regulatory mRNA or protein expression. Alternatively, when expression of cell cycle regulatory mRNA or protein is less (statistically significantly less) in the presence of the candidate compound than in its absence, the candidate compound is identified as an inhibitor of cell cycle regulatory mRNA or protein expression. The level of cell cycle regulatory mRNA or protein expression in the cells can be determined by methods described herein for detecting cell cycle regulatory mRNA or protein.

In preferred embodiments, the cell cycle regulatory protein is a member of the E2F family of transcription factors and the identified compound is a cell cycle checkpoint activation modulator.

The invention further pertains to novel agents identified by the aforementioned screening assays and uses thereof for treatments as described herein.

Pharmaceutical Compositions

Compounds of the present invention, can be incorporated into pharmaceutical compositions suitable for administration. Such compositions typically comprise the compound (i.e. including the active compound), and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In one embodiment, the pharmaceutical composition contains a compound (i.e. active compound) which is a cell cycle checkpoint activation modulator. In another embodiment the active compound of the pharmaceutical composition is identified by the screening assays described herein.

In a preferred embodiment, the pharmaceutical composition contains a compound (i.e. active compound) which is a cell cycle checkpoint activation modulator, where administering the pharmaceutical composition to a subject in need thereof or by contacting a cell with the pharmaceutical composition results in one or more of the following: accumulation of cells in G1 and/or S phase of the cell cycle, cytotoxicity via apoptosis in cancer cells but not in normal cells, antitumor activity in animals with a therapeutic index of at least 2, and modulation of cell cycle checkpoint activation (i.e. elevation of a member of the E2F family of transcription factors).

In more preferred embodiments, the pharmaceutical composition contains a compound (i.e. cell cycle checkpoint activation modulator) that can be 3,4-dihydro-2,2-dimethyl-3-(3-methyl-2-butenyl)-2H-naphtho [1,2-b]pyran-5,6-dione, 3,4-dihydro-2,2-dimethyl-2H-naphtho[1,2-b]thiopyran-5,6-dione or 3,4-dihydro-4,4-dimethyl-2H-naphtho[1,2-b]thiopyran-5,6-dione.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., cell cycle checkpoint activation modulator) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, incorporated fully herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

In therapeutic applications, the dosages of the pharmaceutical compositions used in accordance with the invention vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.0001 mg/kilo per day to about 1000 mg/kilo per day. In preferred embodiments, dosages can range from about 1 mg/kilo per day to about 200 mg/kilo per day. An effective amount of a pharmaceutical agent is that which provides an objectively identifiable improvement as noted by the clinician or other qualified observer. Regression of a tumor in a patient is typically measured with reference to the diameter of a tumor. Decrease in the diameter of a tumor indicates regression. Regression is also indicated by failure of tumors to reoccur after treatment has stopped. As used herein, the terms “dosage effective manner” and “therapeutically effective amount” refers to amount of an active compound to produce the desired effect in a subject or cell.

The pharmaceutical compositions can be included in a container, pack, or dispenser together with instructions for administration.

The invention is further defined by reference to the following examples. It is understood that the foregoing detailed description and the following examples are illustrative only and are not to be taken as limitations upon the scope of the invention. It will be apparent to those skilled in the art that many modifications, both to the materials and methods, may be practiced without departing from the purpose and interest of the invention. Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In the case of conflict, the present specification, including definitions, will control.

### EXAMPLES

### Example 1

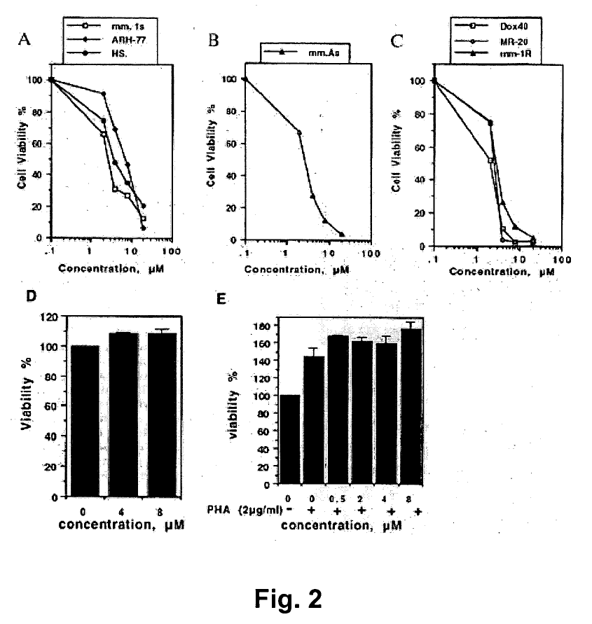

Several studies have shown that β-Lapachone activates checkpoints and induces apoptosis in cancer cells from a variety of tissues without affecting normal cells from these tissues (U.S. Publication No. US-2002-0169135-A1). FIG. 2 shows the differential effects of β-Lapachone on human multiple myeloma (MM) cells vs. normal human Peripheral Blood Mononuclear Cells (PBMC). In this study, proliferation of MM cells cultured in the absence or presence of β-Lapachone (2, 4, 8, and 20 μM) for 24 h was measured by MTT assay. At a concentration of 4 μM, cell viability in cultures was found to be significantly decreased in all seven MM cell lines, including dramatic reduction in the proliferation of a patient's MM cells and drug-resistant cells. To investigate the cytotoxicity of β-Lapachone on human PBMC, cells were isolated from anticoagulant-treated blood. Proliferating PBMC were generated by 72 h incubation with phytohemagglutinin (PHA) at 2 μg/mL. Growth of cells culture in the absence or presence of β-Lapachone (0.5, 2, 4, and 8 μM) for 24 h was measured by MTT. No cytotoxicity to either fresh or proliferating PBMC growth was observed.

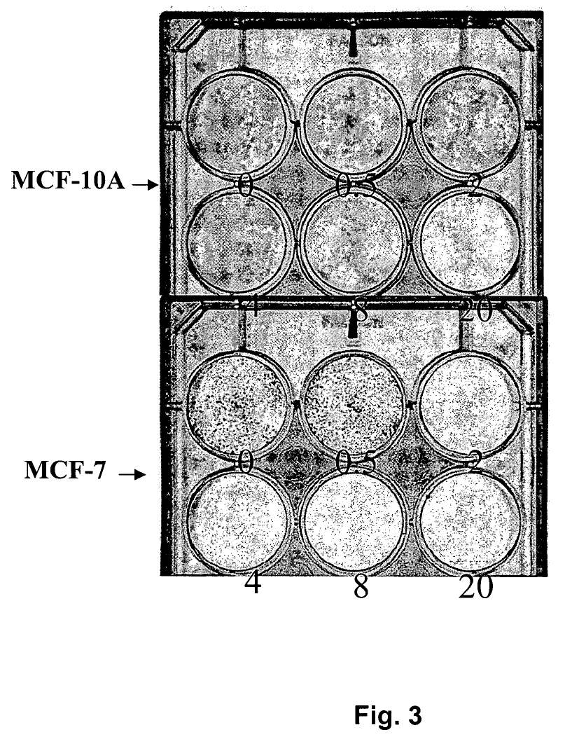

FIG. 3 shows the differential effects of β-Lapachone (μM) on human breast cancer cells (MCF-7) vs. normal human breast epithelial cells (MCF-10A). In this experiment, exponentially growing cells were seeded at 1000 cells/well and allowed to attach for 48 h. The cells were treated for 4 h with β-Lapachone at various concentrations, then were rinsed and fresh medium was added. After 10-20 days, cells were fixed and stained with modified Wright-Giemsa stain. The human breast cancer cells (MCF-7) show essentially complete elimination of colonies at β-Lapachone concentrations of 2-4 μM and higher, whereas the normal breast epithelial cells (MCF-1OA) show no reduction in the number of colonies, although the size of the colonies is smaller, as would be expected by checkpoint activated growth delay.

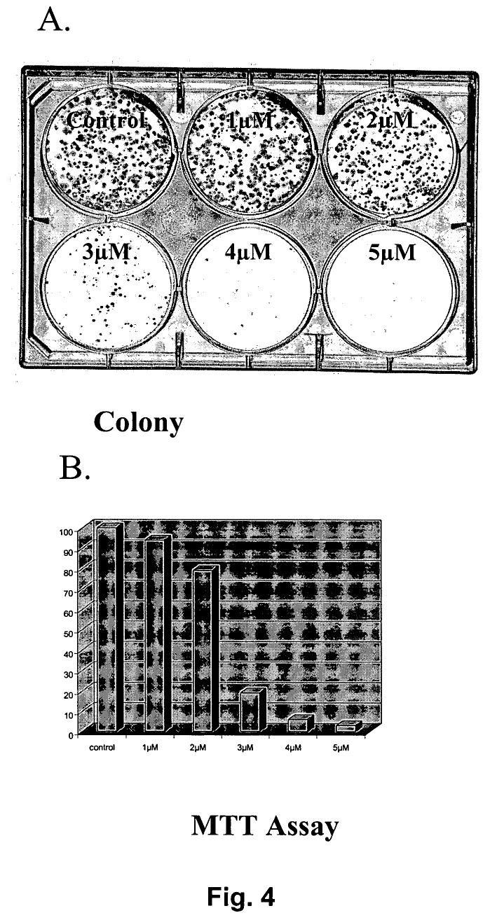

FIG. 4 shows a similar β-Lapachone induced reduction of viability in the human colon cancer cell line DLD1. DLD1 cells were seeded into 6-well, 96-well plates and allowed to attach overnight. Plated cells were then treated with equal volumes of media containing β-Lapachone at various concentrations for 4 h. Control cells were treated with DMSO equivalent to the highest dose of β-Lapachone used. For the colony formation assay, colonies were allowed to grow for 14 days; MTT assay cells continued in culture for an additional 2 days. Both assay methods show that a 4 hour exposure of 4-5 μM β-Lapachone eliminates viable cells.

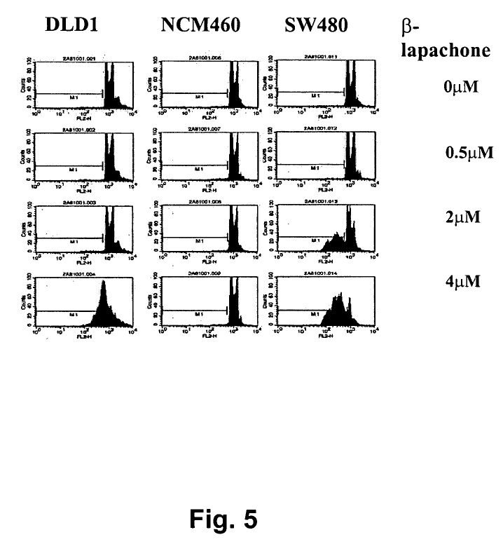

FIG. 5 is a histogram showing that 2-4 μM concentrations of β-Lapachone induce apoptosis in human colon carcinoma cells (DLD1 and SW480) as demonstrated by the appearance of a sub-G1 fraction, whereas no apoptosis is seen in normal human colon cells (NCM460). Cells were treated for 24 h, then were subjected to flow cytometric analysis after staining with propidium iodide.

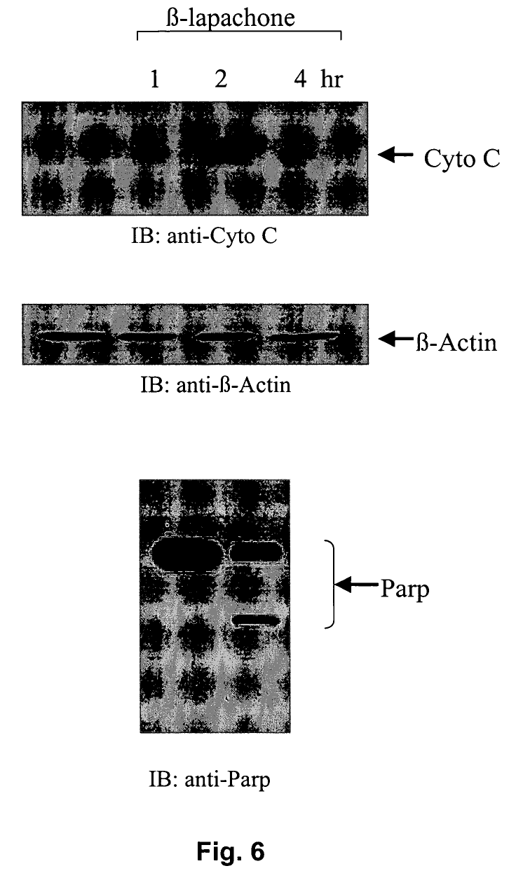

FIG. 6 is a Western blot showing that β-Lapachone stress induces cytochrome c release in DLD1 colon cancer cells after as little as 1 hour of exposure, with release peaking at the 2 hour time point. A second blot in the figure shows the cleavage of PARP after 4 hours of exposure to β-Lapachone. Cytochrome c release and PARP cleavage demonstrates the induction of apoptosis by β-Lapachone.

Similar experiments, as described above with β-Lapachone, were carried out using 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione and 3,4-dihydro-4,4-dimethyl-2H-naphtho[1,2-b]thiopyran-5,6-dione the results of which are described in Table 1. These results show that 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione and 3,4-dihydro-4,4-dimethyl-2H-naphtho[1,2-b]thiopyran-5,6-dione effect cancer cells in a similar manner as β-Lapachone.

TABLE 1

| | | | | | | | | Normal Cell Lines, | | | | | |
|---|---|---|---|---|---|---|---|---|---|---|---|---|---|
| | | Cancer Cell Lines, IC50, μM | | | | | | μM | | | | | |
| | | Prostate | Colon | Breast | Pancreas | Lung | Colon | Colon | Breast | Fold | E2F Induction | | |
| ID | Structure | DLD1 | SW480 | MCF-7 | PaCa-2 | A549 | HT-29 | NCM460 | MCF10A | Selectivity | SW480 | HT-29 | PANC1 |
| β-lapachone | 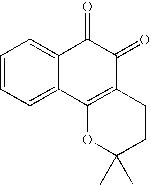 | 3.6 | 3.3 | 1.0 | 1.6 | 1.8 | 2.1 | 8.1 | 5.8 | ˜3 | + | + | + |
| 3,4-dihydro- 2,2- dimethyl-3- (3-methyl-2- butenyl)- 2H- naphtho[1,2- b]pyran-5,6- dione | 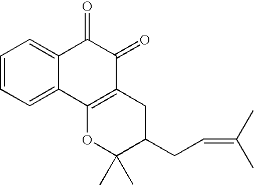 | 1.3 | 1.8 | 1.0 | ND | ND | ND | 10.0 | ND | ˜7 | + | + | + |
| 3.4-dihydro- 2,2- dimethyl- 2H- naphtho[1,2- b]thiopyran- 5.6-dione | 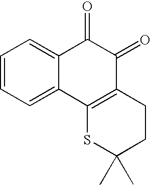 | 2.0 | 1.5 | 1.0 | 1.5 | 1.7 | 1.8 | 7.5 | 6.7 | ˜4.5 | + | + | + |
| 3,4-dihydro- 4,4- dimethyl- 2H- naphtho[1,2- b]thiopyran- 5,6-dione | ND | ND | ND | 1.2 | 1.6 | 1.6 | 6.0 | 6.3 | ˜4 | + | + | + | |

Thus, FIGS. 2-6 and Table 1 show that β-Lapachone, 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione and 3,4-dihydro-4,4-dimethyl-2H-naphtho[1,2-b]thiopyran-5,6-dione, through their interaction with members of the E2F family of transcription factors (i.e. E2F-1, E2F-2, E2F-3) and other cell cycle regulatory proteins, diminishes cell viability and promotes apoptosis in carcinoma cell lines from various tissues without affecting the normal cells from these representative tissues.

### Example 2

A variety of methods are currently available for inducing cell death in cancer cells. However, they all suffer the problem of selectivity as they affect cancer cells and normal cells equally. In a preferred embodiment, the present invention discloses a method, and therapeutic anti-cancer agents, which selectively affect cancer cells without affecting normal cells.

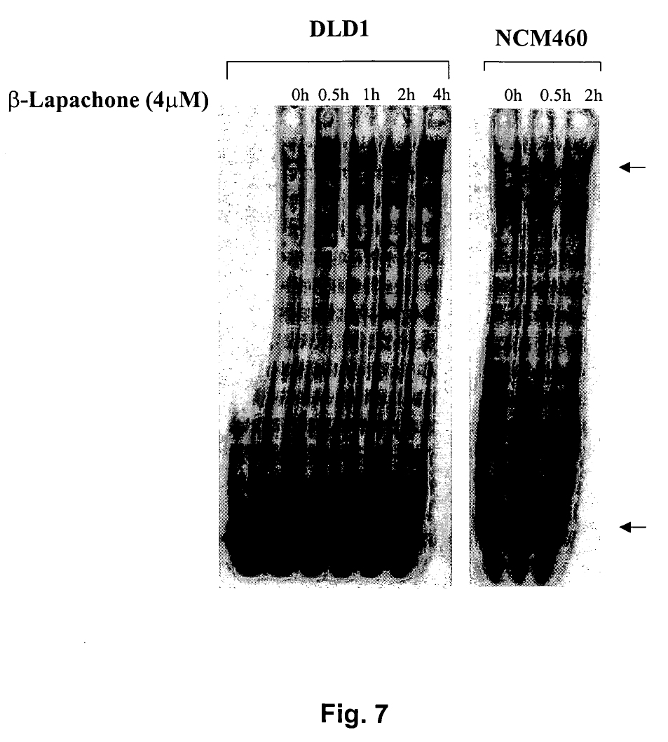

Current methods of inducing E2F involve DNA damage and microtubule stabilization, which is not selective for cancer cells. The studies described in FIGS. 7-11 and Table 1 clearly show the upregulation of members of the E2F family of transcription factors (i.e. E2F-1, E2F-2, E2F-3) in cancer cell lines after treatment with β-Lapachone, 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione and 3,4-dihydro-4,4-dimethyl-2H-naphtho[1,2-b]thiopyran-5,6-dione whereas normal cells are essentially unaffected.

FIG. 7 shows the binding of nuclear proteins from β-Lapachone-treated and -untreated human colon carcinoma cells (LD1) and normal colon cells (NCM460) to a ³²P-labeled, 100-bp, double-stranded DNA subfragment containing three E2F consensus sequences using an gel mobility shift assay. The arrow denotes the location of the putative E2F protein-DNA complex. These results show that the level of E2F expression in the NCM460 normal cells is essentially unchanged after treatment with 4 μM β-Lapachone for up to 2 hours. In contrast, nuclear E2F protein levels are increased in the DLD1 vs. starting levels as early as 0.5 hours after treatment and are markedly elevated after 1 hour of treatment.

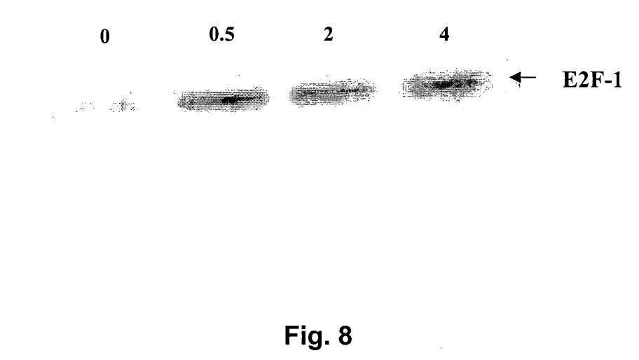

FIG. 8 shows that E2F-1 protein expression is upregulated by β-Lapachone in human pancreatic cancer cells (Paca-2), as demonstrated by Western blot analysis. In this experiment, Paca-2 cells were seeded in medium and exposed for 0.5 hours to 0 (vehicle), 0.5, 2 or 4 μM concentrations of β-Lapachone. Cells were harvested and whole cell lysates were prepared and resolved by SDS/PAGE, then Western blots were prepared using E2F-1 antibody obtained from Santa Cruz Biotechnology (Santa Cruz, Calif.) and an enhanced chemiluminescence assay system (Amersham Pharmacia). The blot shows that E2F-1 protein is induced by the lowest concentration of β-Lapachone tested, 0.5 μM.

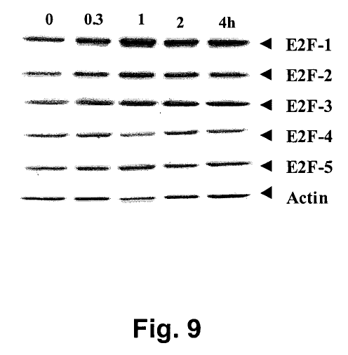

FIG. 9 shows that E2F-1, E2F-2 and E2F-3 protein expression is upregulated by β-Lapachone in human colon cancer cells (SW-480), as demonstrated by Western blot analysis. In this experiment, SW-480 cells were seeded in medium and exposed for 0 to 4.0 hours with 4 μM concentrations of β-Lapachone. Cells were harvested and whole cell lysates were prepared and resolved by SDS/PAGE, then Western blots were prepared using the specific E2F antibodies obtained from Santa Cruz Biotechnology (Santa Cruz, Calif.) and an enhanced chemiluminescence assay system (Amersham Pharmacia). β-Actin was used as a loading control. The blot shows that the expression of E2F-2 and E2F-3 (E2F-1 closely-related family members) occurs during β-lapachone exposure. E2F-4 and E2F-5, which function differently from E2F-1, E2F-2 and E2F-3, are not affected.

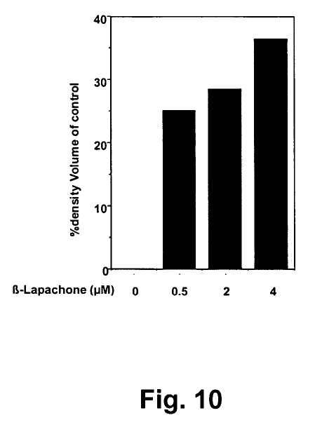

FIG. 10 shows a similar β-Lapachone-induced elevation of E2F-1 levels in colon cancer cells. Human colon cancer cells (SW480) were seeded in medium and exposed to 0.5, 2 or 4 μM β-Lapachone. Cells were harvested and lysate was prepared and analyzed as described in FIG. 6. Relative density of the bands on the blot was measured by gel densitometry. These results show that E2F-1 levels are increased in the SW480 colon cells by 25% following 0.5 hour treatment with 0.5 μM β-Lapachone and up to 35% with 4 μM β-Lapachone.

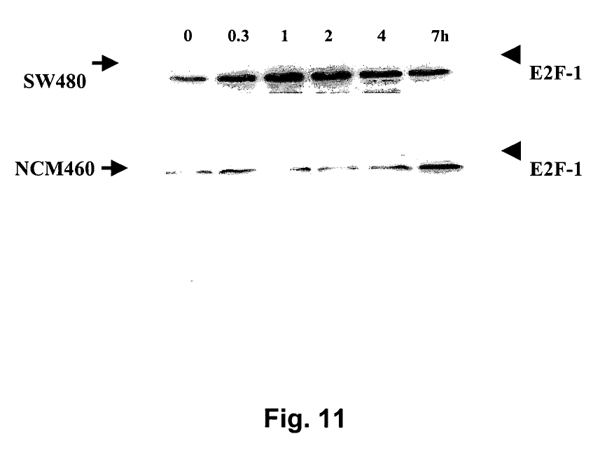

FIG. 11 is a Western blot comparing E2F-1 levels in both colon cancer cells and normal colon cells after β-Lapachone treatment. Human colon cancer cells (SW480) and normal colon cells (NCM460) were seeded in medium and exposed to 2μM β-Lapachone. Cells were harvested prior to treatment and 0.3, 1, 2, 4, or 7 h after exposure and lysate was prepared and analyzed as described in FIG. 6. This experiment shows that E2F-1 induction is observed in the SW480 cells after as little as 0.3 hours β-Lapachone exposure, peaks at 1-2 hours, but is still appreciably elevated at 7 hours, thus demonstrating the persistence of E2F-1 induction in cancer cells. No similar induction of E2F-1 is seen in the NCM460 normal cells.

Similar experiments, as described above with β-Lapachone, were carried out using 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione and 3,4-dihydro-4,4-dimethyl-2H-naphtho[1,2-b]thiopyran-5,6-dione the results of which are described in Table 1. These results show that 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione and 3,4-dihydro-4,4-dimethyl-2H-naphtho[1,2-b]thiopyran-5,6-dione induce members of the E2F family of transcription factors (i.e. E2F-1, E2F-2, E2F-3) in cancer cells.

### Example 3

In addition to Taxol, β-lapachone has been shown to work in combination with other chemotherapeutic agents. In a preferred embodiment, the present invention discloses a method, and therapeutic anti-cancer agents, which selectively affect cancer cells without affecting normal cells, in combination with microtubule targeting drugs, toposiomerase poison drugs and cytidine analogue drugs.

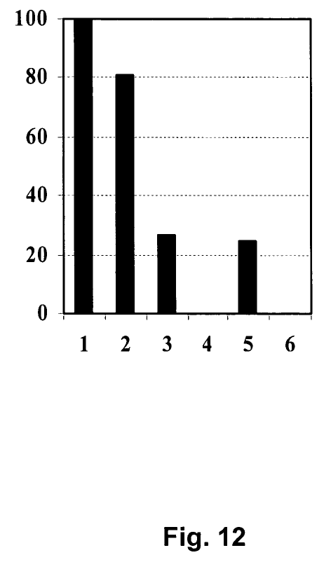

FIG. 12 shows the effectiveness of β-lapachone used in combination with GL331, an analogue of etoposide, which is a topoisomerase II inhibitor. In this experiment, human prostate cancer cells (PC-3) were treated for 4 h with β-lapachone at a concentration of 2 μM and/or GL331 at a concentration of 2 μM. Column 1 shows control cells treated with solvent on days 1 and 2. Column 2 shows cells treated with β-lapachone at 2 μM on day 1 for 4 h, incubated in drug-free medium for 20 h, and then treated with solvent control on day 2. Column 3 shows cells treated with solvent control for 4 h on day 1 and with GL331 at 2 μM for 4 h on day 2. Column 4 shows cells treated with β-lapachone on day 1 and with GL331 on day 2. Column 5 shows cells treated with GL331 on day 1 and with β-lapachone on day 2. Column 6 shows cells treated with β-lapachone and GL331 on day 2. The number of colonies in the control well (solvent-treated) was taken as 100% survival. As shown in the figure, treatment with both drugs simultaneously or treatment with β-lapachone on day 1 followed by GL331 on day 2 resulted in synergistic cytotoxicity and complete eradication of colony forming units. Treatment of cells with β-lapachone following GL331 treatment resulted in to such advantage.

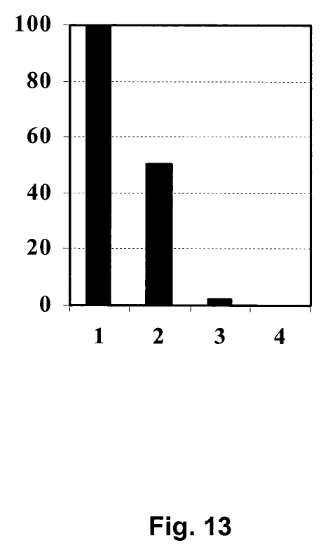

FIG. 13 shows the effectiveness of β-lapachone used in combination with gemcitabine, a cytidine analogue drug. In this experiment, human pancreatic cancer cells (Paca-2) were treated for 4 h with β-lapachone at a concentration of 2 μM and/or gemcitabine at a concentration of 5 μg/ml. Column 1 shows control cells treated with solvent on days 1 and 2. Column 2 shows cells treated with β-lapachone at 2 μM on day 1 for 4 h, incubated in drug-free medium for 20 h, and then treated with solvent control on day 2. Column 3 shows cells treated with solvent for 4 h on day 1 and with gemcitabine at 5 μg/ml for 4 h on day 2. Column 4 shows cells treated with gemcitabine on day 1 and with β-lapachone on day 2. The number of colonies in the control well (solvent-treated) was taken as 100% survival. As shown in the figure, treatment with gemcitabine on day 1 followed by β-lapachone resulted in complete eradication of colony forming units.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a cell proliferative disorder, comprising
administering to a subject in need thereof a therapeutically effective amount of a compound that does not damage DNA and does not stabilize microtubules, and
activating a cell cycle checkpoint in said subject,
wherein said compound is not β-lapachone, and said cell proliferative disorder is treated.

2. The method according to claim 1, wherein said cell proliferative disorder is cancer.

3. The method according to claim 1, wherein said cell proliferative disorder is pre-cancer.

4. The method according to claim 1, wherein said cell cycle checkpoint is a G1 phase or S phase checkpoint.

5. The method according to claim 1, wherein said cell cycle checkpoint is a G1 phase and S phase checkpoint.

6. The method according to claim 1, wherein said cell cycle checkpoint is a G1 phase checkpoint.

7. The method according to claim 1, wherein said cell cycle checkpoint is an S phase checkpoint.

8. The method according to claim 1, wherein said cell cycle checkpoint is a G2 phase or M phase checkpoint.

9. A method of treating a cell proliferative disorder, comprising
administering to a subject in need thereof a therapeutically effective amount of a compound that does not damage DNA and does not stabilize microtubules, and
activating a cell cycle checkpoint pathway in said subject,
wherein said compound is not β-lapachone, and said cell proliferative disorder is treated.

10. The method according to claim 9, wherein said cell proliferative disorder is cancer.

11. The method according to claim 9, wherein said cell proliferative disorder is pre-cancer.

12. The method according to claim 9, wherein said cell cycle checkpoint is a G1 phase or S phase checkpoint.

13. The method according to claim 9, wherein said cell cycle checkpoint is a G1 phase and S phase checkpoint.

14. The method according to claim 9, wherein said cell cycle checkpoint is a G1 phase checkpoint.

15. The method according to claim 9, wherein said cell cycle checkpoint is an S phase checkpoint.

16. The method according to claim 9, wherein said cell cycle checkpoint is a G2 phase or M phase checkpoint.

17. The method according to claim 9, wherein said cell cycle checkpoint pathway comprises a protein.

18. The method according to claim 9, wherein said cell cycle checkpoint pathway comprises a molecule that is not a protein.

19. The method according to claim 9, wherein said cell cycle checkpoint pathway comprises an E2F pathway.

20. A method of treating a cell proliferative disorder, comprising
administering to a subject in need thereof a therapeutically effective amount of a compound that does not damage DNA and does not stabilize microtubules, and
activating a cell cycle checkpoint molecule in said subject,
wherein said compound is not β-lapachone, and said cell proliferative disorder is treated.

21. The method according to claim 20, wherein said cell proliferative disorder is cancer.

22. The method according to claim 20, wherein said cell proliferative disorder is pre-cancer.

23. The method according to claim 20, wherein said cell cycle checkpoint is a G1 phase or S phase checkpoint.

24. The method according to claim 20, wherein said cell cycle checkpoint is a G1 phase and S phase checkpoint.

25. The method according to claim 20, wherein said cell cycle checkpoint is a G1 phase checkpoint.

26. The method according to claim 20, wherein said cell cycle checkpoint is an S phase checkpoint.

27. The method according to claim 20, wherein said cell cycle checkpoint is a G2 phase or M phase checkpoint.

28. The method according to claim 20, wherein said cell cycle checkpoint molecule is a protein.

29. The method according to claim 20, wherein said cell cycle checkpoint molecule is not a protein.

30. The method according to claim 20, wherein said cell cycle checkpoint molecule comprises an E2F pathway molecule.

31. The method according to claim 20, wherein said cell cycle checkpoint molecule comprises E2F-1, E2F-2, or E2F-3.

32. The method according to claim 20, wherein said cell cycle checkpoint molecule comprises E2F-1.

33. The method according to claim 20, wherein said cell cycle checkpoint molecule comprises E2F-2.

34. The method according to claim 20, wherein said cell cycle checkpoint molecule comprises E2F-3.

35. A method of treating a cell proliferative disorder, comprising
administering to a subject in need thereof a therapeutically effective amount of a compound that does not damage DNA and does not stabilize microtubules, and
stimulating unscheduled expression of a cell cycle checkpoint molecule in said subject,
wherein said compound is not β-lapachone, and said cell proliferative disorder is treated.

36. The method according to claim 35, wherein said cell proliferative disorder is cancer.

37. The method according to claim 35, wherein said cell proliferative disorder is pre-cancer.

38. The method according to claim 35, wherein said cell cycle checkpoint molecule is a G1 phase or S phase checkpoint molecule.

39. The method according to claim 35, wherein said cell cycle checkpoint molecule is a G1 phase and S phase checkpoint molecule.

40. The method according to claim 35, wherein said cell cycle checkpoint molecule is a G1 phase checkpoint molecule.

41. The method according to claim 35, wherein said cell cycle checkpoint molecule is an S phase checkpoint molecule.

42. The method according to claim 35, wherein said cell cycle checkpoint molecule is a G2 phase or M phase checkpoint molecule.

43. The method according to claim 35, wherein said cell cycle checkpoint molecule is a protein.

44. The method according to claim 35, wherein said cell cycle checkpoint molecule is not a protein.

45. The method according to claim 35, wherein said cell cycle checkpoint molecule comprises an E2F pathway molecule.

46. The method according to claim 35, wherein said cell cycle checkpoint molecule comprises E2F-1, E2F-2, or E2F-3.

47. The method according to claim 35, wherein said cell cycle checkpoint molecule comprises E2F-1.

48. The method according to claim 35, wherein said cell cycle checkpoint molecule comprises E2F-2.

49. The method according to claim 35, wherein said cell cycle checkpoint molecule comprises E2F-3.

50. A method of treating a cell proliferative disorder, comprising
administering to a subject in need thereof a therapeutically effective amount of a compound that does not damage DNA and does not stabilize microtubules, and
activating an E2F pathway in said subject,
wherein said compound is not β-lapachone, and said cell proliferative disorder is treated.

51. The method according to claim 50, wherein said cell proliferative disorder is cancer.

52. The method according to claim 50, wherein said cell proliferative disorder is pre-cancer.

53. The method according to claim 50, wherein said E2F pathway comprises a protein.

54. The method according to claim 50, wherein said E2F pathway comprises a molecule that is not a protein.

55. The method according to claim 50, wherein said activating an E2F pathway comprises elevating the level of an E2F pathway molecule.

56. The method according to claim 55, wherein said E2F pathway molecule comprises E2F-1, E2F-2, or E2F-3.

57. The method according to claim 50, wherein said activating an E2F pathway comprises increasing the activity of an E2F pathway molecule.

58. The method according to claim 57, wherein said E2F pathway molecule comprises E2F-1, E2F-2, or E2F-3.

59. The method according to claim 50, wherein said activating an E2F pathway comprises elevating the level of E2F-1.

60. The method according to claim 50, wherein said activating an E2F pathway comprises elevating the level of E2F-2.

61. The method according to claim 50, wherein said activating an E2F pathway comprises elevating the level of E2F-3.

62. A method of treating a cell proliferative disorder, comprising
administering to a subject in need thereof a therapeutically effective amount of a compound that does not damage DNA and does not stabilize microtubules, and
elevating the level of a member of the E2F family of transcription factors in said subject,
wherein said compound is not β-lapachone, and said cell proliferative disorder is treated.

63. The method according to claim 62, wherein said cell proliferative disorder is cancer.

64. The method according to claim 62, wherein said cell proliferative disorder is pre-cancer.

65. The method according to claim 62, wherein said member of the E2F family of transcription factors comprises E2F-1, E2F-2, or E2F-3.

66. The method according to claim 62, wherein said member of the E2F family of transcription factors comprises E2F-1.

67. The method according to claim 62, wherein said member of the E2F family of transcription factors comprises E2F-2.

68. The method according to claim 62, wherein said member of the E2F family of transcription factors comprises E2F-3.

69. A method of treating cancer, comprising
administering to a subject in need thereof a therapeutically effective amount of a compound that does not damage DNA and does not stabilize microtubules, and
activating a cell cycle checkpoint in said subject,
wherein said compound is not β-lapachone, and said cancer is treated.

70. The method according to claim 69, wherein said cell cycle checkpoint is a G1 phase or S phase checkpoint.

71. The method according to claim 69, wherein said cell cycle checkpoint is a G1 phase and S phase checkpoint.

72. The method according to claim 69, wherein said cell cycle checkpoint is a G1 phase checkpoint.

73. The method according to claim 69, wherein said cell cycle checkpoint is an S phase checkpoint.

74. The method according to claim 69, wherein said cell cycle checkpoint is a G2 phase or M phase checkpoint.

75. A method of treating cancer, comprising
administering to a subject in need thereof a therapeutically effective amount of a compound that does not damage DNA and does not stabilize microtubules, and
stimulating unscheduled expression of a cell cycle checkpoint molecule in said subject,
wherein said compound is not β-lapachone, and said cancer is treated.

76. The method according to claim 75, wherein said cell cycle checkpoint molecule is a G1 phase or S phase checkpoint molecule.

77. The method according to claim 75, wherein said cell cycle checkpoint molecule is a G1 phase and S phase checkpoint molecule.

78. The method according to claim 75, wherein said cell cycle checkpoint molecule is a G1 phase checkpoint molecule.

79. The method according to claim 75, wherein said cell cycle checkpoint molecule is an S phase checkpoint molecule.

80. The method according to claim 75, wherein said cell cycle checkpoint molecule is a G2 phase or M phase checkpoint molecule.

81. The method according to claim 75, wherein said cell cycle checkpoint molecule is a protein.

82. The method according to claim 75, wherein said cell cycle checkpoint molecule is not a protein.

83. The method according to claim 75, wherein said cell cycle checkpoint molecule comprises an E2F pathway molecule.

84. The method according to claim 75, wherein said cell cycle checkpoint molecule comprises E2F-1, E2F-2, or E2F-3.

85. The method according to claim 75, wherein said cell cycle checkpoint molecule comprises E2F-1.

86. The method according to claim 75, wherein said cell cycle checkpoint molecule comprises E2F-2.

87. The method according to claim 75, wherein said cell cycle checkpoint molecule comprises E2F-3.

88. A method of treating or preventing an apoptosis-related disorder in a subject, comprising
administering to a subject in need thereof a therapeutically effective amount of a compound that does not damage DNA and does not stabilize microtubules, and
activating a cell cycle checkpoint in said subject,
wherein said compound is not β-lapachone, and said apoptosis-related disorder is treated or prevented.

89. A method according to claim 88, wherein said apoptosis-related disorder is arthritis, inflammation, or autoimmune disease.

90. The method according to claim 88, wherein said cell cycle checkpoint is a G1 phase or S phase checkpoint.

91. The method according to claim 88, wherein said cell cycle checkpoint is a G1 phase and S phase checkpoint.

92. The method according to claim 88, wherein said cell cycle checkpoint is a G1 phase checkpoint.

93. The method according to claim 88, wherein said cell cycle checkpoint is an S phase checkpoint.

94. The method according to claim 88, wherein said cell cycle checkpoint is a G2 phase or M phase checkpoint.

95. A method of treating or preventing an apoptosis-related disorder in a subject, comprising
administering to a subject in need thereof a therapeutically effective amount of a compound that does not damage DNA and does not stabilize microtubules, and
stimulating unscheduled expression of a cell cycle checkpoint molecule in said subject,
wherein said compound is not β-lapachone, and said apoptosis-related disorder is treated or prevented.

96. A method according to claim 95, wherein said apoptosis-related disorder is arthritis, inflammation, or autoimmune disease.

97. The method according to claim 95, wherein said cell cycle checkpoint molecule is a G1 phase or S phase checkpoint molecule.

98. The method according to claim 95, wherein said cell cycle checkpoint molecule is a G1 phase and S phase checkpoint molecule.

99. The method according to claim 95, wherein said cell cycle checkpoint molecule is a G1 phase checkpoint molecule.

100. The method according to claim 95, wherein said cell cycle checkpoint molecule is an S phase checkpoint molecule.

101. The method according to claim 95, wherein said cell cycle checkpoint molecule is a G2 phase or M phase checkpoint molecule.

102. The method according to claim 95, wherein said cell cycle checkpoint molecule is a protein.

103. The method according to claim 95, wherein said cell cycle checkpoint molecule is not a protein.

104. The method according to claim 95, wherein said cell cycle checkpoint molecule comprises an E2F pathway molecule.

105. The method according to claim 95, wherein said cell cycle checkpoint molecule comprises E2F-1, E2F-2, or E2F-3.

106. The method according to claim 95, wherein said cell cycle checkpoint molecule comprises E2F-1.

107. The method according to claim 95, wherein said cell cycle checkpoint molecule comprises E2F-2.

108. The method according to claim 95, wherein said cell cycle checkpoint molecule comprises E2F-3.
